(19) United States
(12) Patent Application Publication
(10) Pub. No.: US 2007/0106229 A1
(43) Pub. Date: May 10, 2007

(54) Safety Intravenous Injection Device

(76) Inventor: Kuang-Chung Wong, Taipei (TW)

Correspondence Address: LIN & ASSOCIATES INTELLECTUAL PROPERTY, P.O. BOX 2339, SARATOGA, CA, 95070-0339, US

(21) Appl. No.: 11/558,468

(22) Filed: Nov. 10, 2006

(30) Foreign Application Priority Data
Nov. 10, 2005 (TW) 094219692

(51) Int. Cl. A61M 5/00

(52) U.S. Cl. 604/249

(57) ABSTRACT

A safety intravenous injection device comprises an injection port, a guiding device, and an injection valve. The injection port comprises an injection tube, a slanting branch tube, and a step-rim seat. The injection tube is in fluid communication with the seat and the branch tube. The injection valve made of silica gel is disposed in the seat. A central part of the injection valve has a plurality of slits and is divided into a plurality of flap portions. The flap portions are free to flex under pressure, and resumes to an original state after pressure is relieved. Lower parts of the flap portions have downward convex curves. The guiding device has a sleeve section engaged with a step rim of the seat and a short tube having a lug on each of the two sides of its opening. The safety intravenous injection device may further comprise a lid.

### BACKGROUND OF THE INVENTION

1. Field of the Invention

The present invention relates to an injection device for an intravenous drip or a blood transfusion, and in particular to a safety intravenous injection device that prevents the backflow of the medication during the injection. Because the injection port is provided with a lid, it not only keeps the dust out, but also avoids the parts of the device being lost.

2. The Prior Arts

The conventional safety intravenous injection device comprises a transmitting tube. Each of the two ends of the transmitting tube has a needle to insert into a vein and a container (glass bottle, plastic bottle and plastic bag), respectively. The end of the transmitting tube near the container has a monitoring bottle and the end near the human body has an injection port, which is used to add a dose of medicine with a syringe in the process of dripping.

FIG. 8 is a schematic view showing a conventional injection port A. One end of the injection port A is an injection inlet A1 and the other end opposite to the injection inlet A1 is a port outlet A2 connecting with a transmitting tube B. The injection port A is in Y-shaped and provided with an slanting branch tube A3 connecting with a transmitting tube B that allows the fluid coming from the container to go through the injection port A and be drained out of port outlet A2. A diaphragm D is bound by a cuff C and seals the injection inlet A1.

The diaphragm D is pierced by a needle E1 of a syringe E and medicinal fluid is injected into the injection port A to enter the patient's body. The diaphragm D is made of a material, such as rubber, and seals once the needle E1 is removed.

### SUMMARY OF THE INVENTION

The diaphragm D pierced by the needle E1 as mentioned above has the following disadvantages:

First of all, when the needle E1 pierces the diaphragm D, the injection port A needs to be held by one hand and the syringe E needs to be held by the other hand. It is likely that the needle E1 accidentally punctures the health provider's skin. Accidental impalement of the health care provider is of grave concern for the transmission of infectious diseases, especially HIV and hepatitis.

Secondly, when the needle E1 pierces the diaphragm D, it may produce rubber chippings, which will flow into the patient's vein and cause damage.

Thirdly, when the intravenous drip is required over a prolonged period of time and the medicinal fluids are required to be injected through the injection port A, repeated piercing of the daiphragm D produces a plurality of pinholes thereon. The pinholes are enlarged and the elastic diaphragm D cannot seal itself. It results in a dripping leak. Even bacteria or virus enters the safety intravenous catheter through the pinholes of the diaphragm D and causes infection.

A primary objective of the present invention is to provide an injection port comprising an injection tube, a branch tube and a step-rim seat. The step-rim seat is provided at one end of the injection tube and includes a through hole in the center. A hollow central portion of the injection tube and the through hole of the step-rim seat are a through passage. The injection tube is in fluid communication with the branch tube.

Another objective of the present invention is to provide an injection valve that forms a fluid-tight seal disposed in the through hole of the step-rim seat. A central part of the injection valve has a plurality of slits and is divided into a plurality of flap portions. The slits between the flap portions are separated interfaces, so every flap portion is free to flex under pressure and resumes to an original state after the pressure is relieved. The injection valve has a sidewall. The flap portions are parted to provide a passage for a needle or a tip of syringe to pass through when the flap portions are pressed downward by the syringe. Lower parts of the flap portions have downward convex curves.

A further objective of the present invention is to provide a guiding device comprising a sleeve section mounted on the step rim of the seat, and a short tube. An interior space is formed at the center of the sleeve section and the seat and the valve is disposed therein. The other side of the guiding device has the short tube for syringe insertion. Both sides of an opening of the short tube are provided with a lug, respectively. The short tube allows the insertion of a syringe alone or a syringe with a needle. The syringe may be a Luer lock syringe (used in United States) or a Friction lock syringe (used in Asia). The needle maybe a metal needle or a plastic needle. The through hole of the guiding device passes through the short tube and the sleeve section. Therefore, the guiding device, the injection valve and the injection tube have a through passage to pass through all three parts.

A still further objective is fulfilled by a second preferred embodiment of the present invention. The guiding device and the injection valve are not changed and a small change is made to the injection port. The lower step rim of the seat of the injection port further comprises a projecting section having a through hole at the center thereof. A lid is provided to cover the top of the guiding device. The lid is in a circular or rectangular shape and has a brim. A long lid loop made of rubber or a highly flexible material is extended from the sidewall of the lid. The free end of the lid loop may be shaped in a sphere or a cone. It passes through and is fixed at the through hole at the projecting section. Inner side of the lid may further comprise two buckle lugs facing each other at the rim. An engaging mortise is form between the buckle lug and the lid. The lugs of the guiding device are inserted into the engaging mortises and rotated to engage with the buckle lugs.

When the medicinal fluid injection is needed, open the lid without detaching the lid loop from the step rim of the seat. Taking advantage of the flexibility of the lid loop, the lid can be pushed aside to reveal the opening of the guiding device and the needle or the syringe tip can be aimed at and inserted into the guiding device to inject the medicine.

When the needle or the syringe tip is inserted into the opening of the guiding device and passes through the flap portions of the injection valve, the flap portions are flexed downward and provide a tight passage for the needle or the tip of the syringe due to its flexibility. Thus the injection goes smoothly. When the needle or the syringe tip is withdrawn after the injection, the flap portions resumes to its original state.

The present invention overcomes the disadvantages of the conventional injection method that needs to pierce the diaphragm on the injection port. It prevents the health care provider from being accidentally punctured by the needle. The injection port according to the present invention prevents producing rubber chippings and pinhole leaking due to enlarged pinholes after repeated injections. Moreover, the lid in accordance with the present invention can prevents the dust from entering the catheter through the guiding device.

### BRIEF DESCRIPTION OF THE DRAWINGS

The present invention will be apparent to those skilled in the art by reading the following detailed description of preferred embodiments thereof, with reference to the attached drawings, in which:

FIG. 1 is an exploded view showing a safety intravenous injection device according to a first preferred embodiment of the present invention;

FIG. 2 is an assembled view showing the safety intravenous injection device according to the first preferred embodiment of the present invention;

FIG. 3 shows an injection valve according to the first preferred embodiment of the present invention;

FIG. 4 shows the safety intravenous injection device according to the first preferred embodiment of the present invention in use;

FIG. 5 is an exploded view showing a safety intravenous injection device according to a second preferred embodiment of the present invention;

FIG. 6 is an assembled view showing the safety intravenous injection device according to the second preferred embodiment of the present invention;

FIG. 7 shows the safety intravenous injection device according to the second preferred embodiment of the present invention in use;

FIG. 8 is an assembled view showing a conventional intravenous injection device in use.

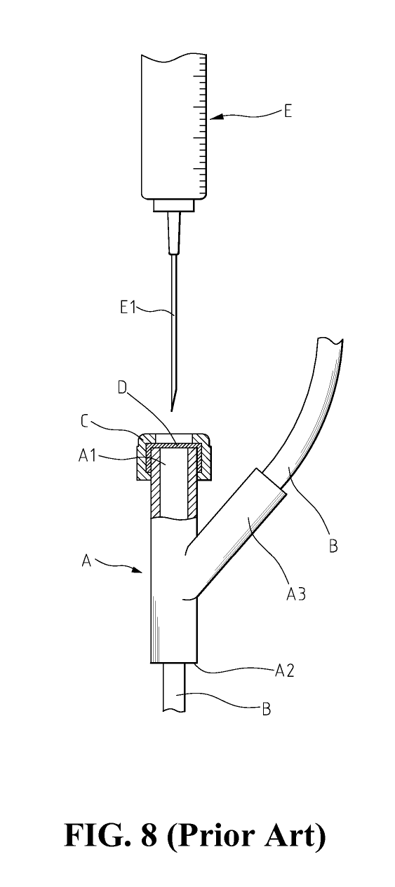

### DETAILED DESCRIPTION OF THE PREFERRED EMBODIMENT

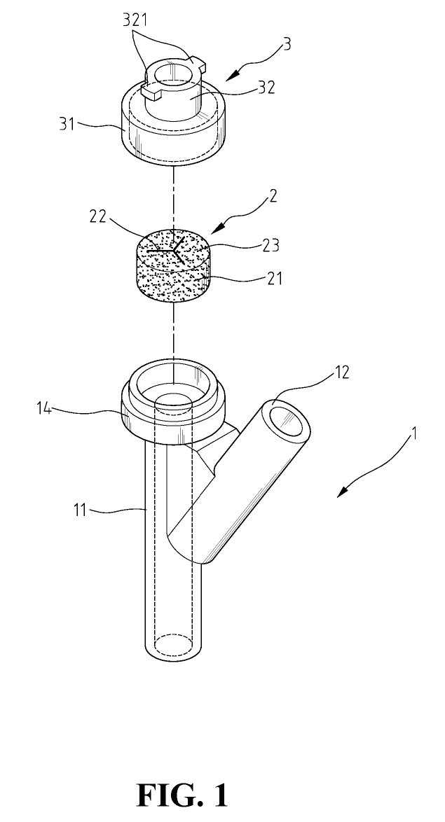

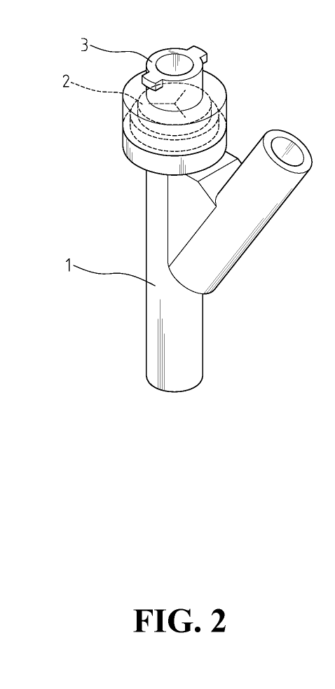

Referring to FIGS. 1 and 2, a safety intravenous injection device according to a first embodiment of the present invention comprises an injection port 1, an injection valve 2 and a guiding device 3.

The injection port 1 comprises an injection tube 11, a slanting branch tube 12, and a step-rim seat 14. The seat 14 is provided at one end of the injection tube 11. The seat 14 is a ring structure whose central hollow portion is in fluid communication with the hollow portion of the injection tube 11. The injection tube 11 and the branch tube 12 are a Y-shaped tube and in fluid communication. The injection port 1 may be integrally molded or glue fixed together by separated components. The injection tube 11 and the branch tube 12 may be in a circular, a rectangular or other geometric shape.

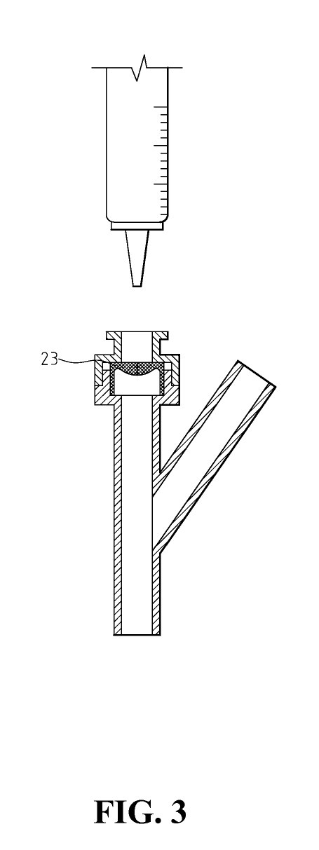

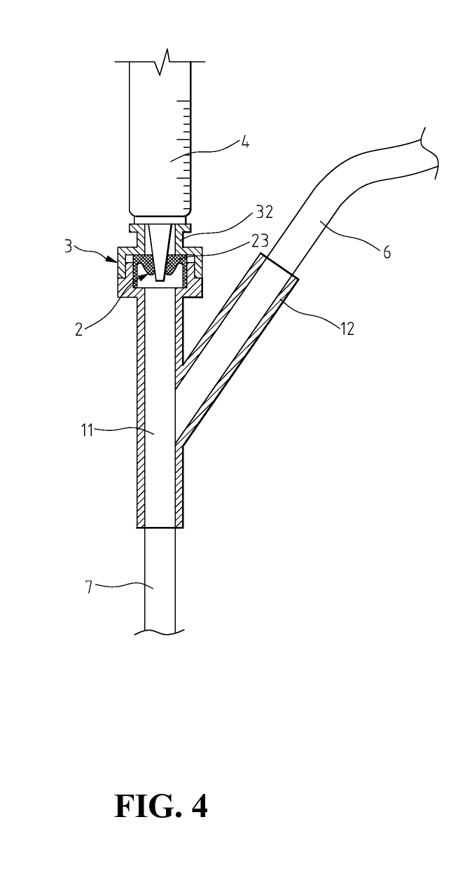

The guiding device 3 comprises a sleeve section 31 and a short tube 32. The interior of the guiding device 3 is a through hole. When the sleeve section 31 is mounted on the step rim of the seat 14, an interior space is formed at the center of the sleeve section 31 and the seat 14. The jointing of the sleeve section 31 and the seat 14 can be direct engagement or glue-fixed. The short tube 32 is disposed on the top of the sleeve section 31 and in fluid communication with it. Both sides of the short tube opening are provided with a lug 321, respectively. The short tube 32 is suitable to insert a plastic needle, a metal needle, a Luer lock syringe (used in United State) or a Friction lock syringe (used in Asia) to inject medicinal fluids into the opening of the guiding device 3. The lugs 321 are provided for the Luer lock syringe having a rotational engagement device (not shown in figures). When the tip of the Luer lock syringe is inserted into the opening of the guiding device 3, the engagement device of the syringe rotates against and engages with the lug 321. Thus the syringe and the guiding device 3 are connected together. During the intravenous dripping, a pressurizer (not shown in figures) will be disposed outside of the container to force the fluid to enter the vein. Because the conventional safety intravenous device can only sustain a pressure of 300 mmHg, the device has a problem of the fluid backflow. For this reason, the safety intravenous injection device according to the present invention provides the injection valve 2 to form a fluid-tight seal. The injection valve 2 is made of silica gel and disposed between the injection port 1 and the guiding device 3. The injection valve 2 having a sidewall 21 engages with the seat 14. The way of the injection valve 2 engaging with the seat 14 may be direct insertion or glue-fixed. A central part of the injection valve 2 has a plurality of slits 22 and is divided into a plurality of flap portions 23. The slits 22 can be in Y, or X shape. The slits 22 separate the injection valve 2 into the flap portions 23. The sealable interfaces between the flap portions 23 are freely movable. As shown in FIG. 3, the lower part of the flap portion 23 has a downward convex curve. The convex shape tends to enhance the fluid tightness of the valve 2 in that it increases its resistance to fluid backflow from the proximal side of the valve 2. The convex shape also tends to cause the tip of the syringe placed against the valve 2 to center on the valve 2 and to be easier to insert through the valve 2 to inject the fluid therein without use of a sharp. As shown in FIG. 4, every flap portions 23 can flex downward under the pressure and provide a passage due to its original characteristic of the material. The material of the valve 2 is tough, so that it does not produce chippings after repeated piercing of the needle or the syringe tip.

FIG. 4 shows the safety intravenous injection device according to the first embodiment of the present invention in use. One end of a catheter 6 connects with the branch tube 12 and the other end of the catheter 6 connects with the container or a medicine bag (not shown in the figures). The free end of the injection tube 11 connects with another catheter 7. The other end of the catheter 7 connects with an injection needle positioning in the human body (not shown in the figures). When the syringe 4 is inserted into the safety intravenous injection device, the needle or the syringe tip is inserted into the short tube 32 of the guiding device 3, thereby reaching the injection valve 2. The injection valve 2 is slit to form the flap portions 23. When the needle or the syringe tip is push downward and reaches the flap portions 23, they will flex downward to form a passage for the needle or the syringe tip to pass through. Thus the medicine is injected into the safety intravenous injection device. The catheter 7 receives the medicinal fluid from the injection tube 11 and the intravenous fluid from the branch tube 12. When the syringe 4 is removed after the injection, the flap portions 23 returns to their original state. The intravenous injection device is sealed to keep the medicine from flowing back.

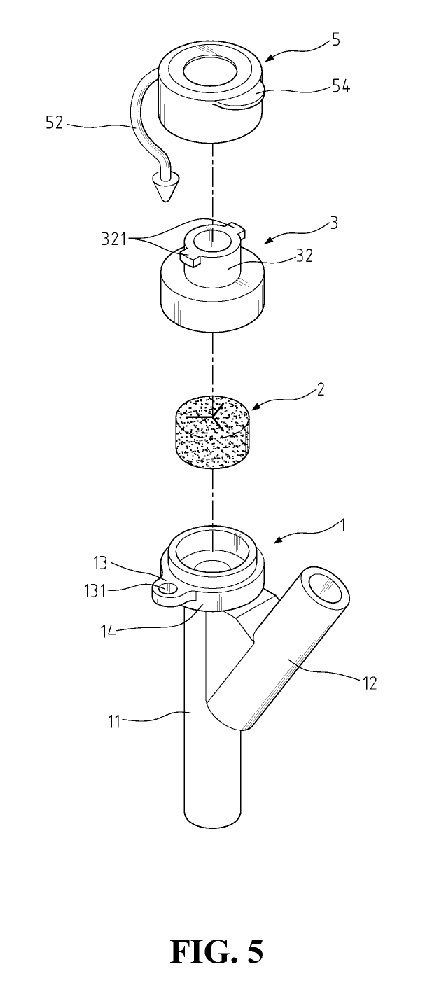

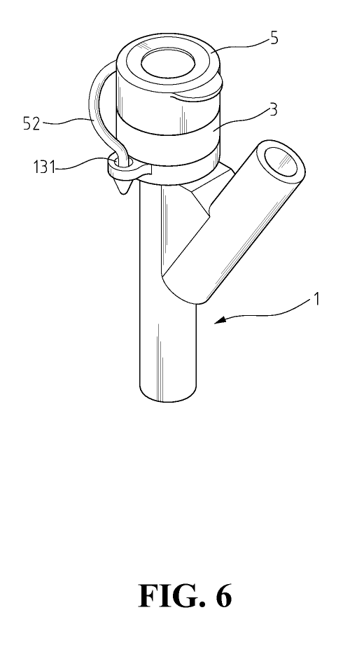

Referring to FIGS. 5 to 7, a safety intravenous injection device in accordance with a second preferred embodiment of the present invention comprises an injection port 1, an injection valve 2 and a guiding device 3 as the first embodiment. However, the lower step rim of the seat 14 of the injection port 1 according to the second preferred embodiment further comprises a projecting section 13. The projecting section 13 has a through hole 131 at the center thereof. A lid 5 is capped at the top of the guiding device 3 directly or by a tenon-mortise device. For the tenon-mortise device, an inner side of the lid 5 may further include two buckle lugs 53 facing each other at the rim. An engaging mortise is form between the buckle lug 53 and the lid 5. The lugs 321 of the guiding device 3 are inserted into the engaging mortises of the lid 5 and rotated to engage with the buckle lugs 53, whereby the lid 5 is capped on the guiding device 3. The lid 5 has a circular or rectangular shape and a lid brim 54. The sidewall of the lid 5 includes an extended lid loop 52 made of rubber or a highly flexible material. The free end of the lid loop 52, having a sphere or a cone shape, passes through and fixed at the through hole 131 of the projecting section 13. The projecting section may be in a hook-shape structure, and has a narrow gap between a tip of the hook and the step rim of the seat 14. The lid loop 52 is squeezed through the narrow gap between the tip of the hook and the step rim of the seat 14 and fixed at the through hole 131 surrounded by the hook.

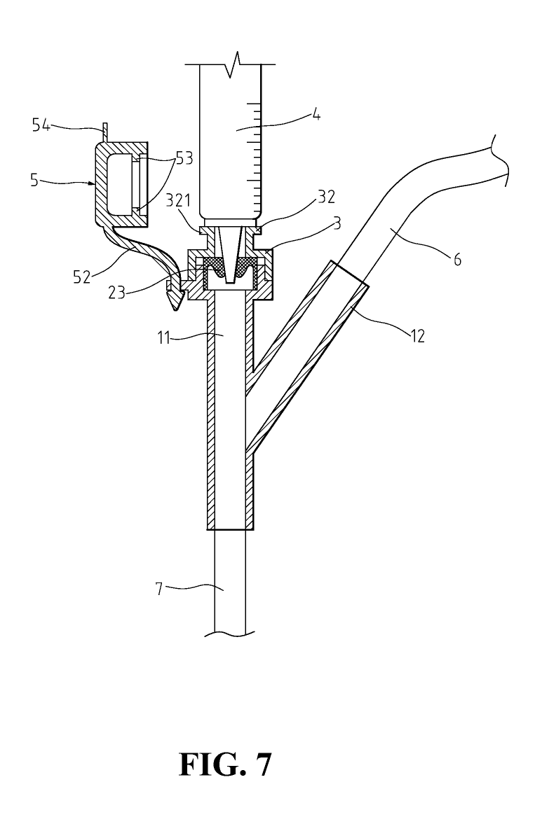

FIG. 7 shows the safety intravenous injection device according to the second embodiment in use. When the patient needs injection, flip the lid 5 from the guiding device 3. Due to the flexibility of the lid loop 52, the free end of the lid loop 52 and the projecting section 13 does not need to be taken apart. Just push the lid 5 asides directly to reveal the short tube 32. Aim the needle or the syringe tip at the opening of the guiding device 3 and insert it through the passage formed between the flap portions 23 to inject the medicinal fluid. The catheter 7 delivers the medicinal fluid from the injection tube 11 and the intravenous fluid from the branch tube 12 to the vein.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A safety intravenous injection device, comprising
an injection port including an injection tube, a slanting branch tube, and a step-rim seat disposed at one end of the injection tube, wherein the injection tube is in fluid communication with the branch tube and the seat;
a guiding device including a sleeve section mounted on a step rim of the seat, and a short tube disposed on top of the sleeve section and in fluid communication with the sleeve section, wherein both sides of an opening of the short tube are provided with a lug, respectively; and
an injection valve made of silica gel having a sidewall and embedded into the seat, wherein a central part of the injection valve has a plurality of slits and is divided into a plurality of flap portions, wherein the slits between the flap portion are separated interface, so that every flap portion is free to flex individually; and lower parts of the flap portions have downward convex curves.

2. The injection device as claimed in claim 1, wherein the injection tube and the branch tube are circular tubes.

3. The injection device as claimed in claim 1, wherein the injection tube and the branch tube are rectangular tubes.

4. The injection device as claimed in claim 1, wherein the slits on the injection valve are in a Y shape.

5. The injection device as claimed in claim 1, wherein the slits on the injection valve are in an X shape.

6. The injection device as claimed in claim 1, wherein the sleeve section and the seat are sleeve-joined.

7. The injection device as claimed in claim 1, wherein the sleeve section and the seat are glue-joined.

8. The injection device as claimed in claim 1, wherein the injection tube and the seat are one of an integral part and separated parts glue-joined.

9. The injection device as claimed in claim 1, wherein the injection valve and the seat are one of directly embedded and glue-joined.

10. A safety intravenous injection device, comprising
an injection port including an injection tube, a slanting branch tube, and a step-rim seat disposed at one end of the injection tube, wherein a lower step rim of the seat includes a projecting section having a through hole at the center thereof, wherein the injection tube is in fluid communication with the branch tube and the seat;
a guiding device including a sleeve section mounted on a step rim of the seat, and a short tube disposed on top of the sleeve section and in fluid communication with the sleeve section, wherein both sides of an opening of the short tube are provided with a lug, respectively;
an injection valve having a sidewall and embedded into the seat, wherein a central part of the injection valve has a plurality of slits and is divided into a plurality of flap portions, wherein the slits between the flap portion are separated interface, so that every flap portion is free to flex individually; lower parts of the flap portions have downward convex curves; and
a lid having a lid loop extended from a sidewall thereof, wherein the free end of the lid loop connects with the through hole of the projecting section.

11. The injection device as claimed in claim 10, wherein the lid has a lid brim and one of a circular shape and a rectangular shape.

12. The injection device as claimed in claim 10, wherein an inner side of the lid has two buckle lugs facing each other at a rim; and wherein an engaging mortise is formed between the buckle lug and the lid

13. The injection device as claimed in claim 10, wherein the free end of the lid loop is in a cone shape.

14. The injection device as claimed in claim 10, wherein the free end of the lid loop is in a sphere shape.
